(19) United States
(12) Patent Application Publication
(10) Pub. No.: US 2008/0027459 A1
(43) Pub. Date: Jan. 31, 2008

(54) Implant integrity measurement apparatus

(75) Inventor: Bret A. Ferree, Cincinnati, OH (US)

Correspondence Address: O''Melveny & Myers LLP;IP&T Calendar Department LA-1118, 400 South Hope Street, Los Angeles, CA, 90071-2899, US

(73) Assignee: Anova Corp.

(21) Appl. No.: 11/880,848

(22) Filed: Jul. 23, 2007

Related U.S. Application Data
(62) Division of application No. 10/410,026, filed on Apr. 9, 2003, now abandoned.
(60) Provisional application No. 60/371,377, filed on Apr. 10, 2002.

(51) Int. Cl. A61B 17/58

(52) U.S. Cl. 606/102

(57) ABSTRACT

Instrumentation for determining the integrity of a natural or synthetic implant includes a component that is at least temporarily attached to or constrained by a bone or tissue, and a handle coupled to the component including a force-measurement gauge. Operation of the device permits a user to push or pull on the handle and determine the integrity of the component by referring to the force-measurement gauge. The handle may be modularly attachable to a plurality of different components or the same component at different sizes for different uses. One preferred embodiment resides in a disc pressure gauge that helps in determining the risk of extrusion of intradiscal devices. Although described in terms of spinal applications, the invention is not limited in this regard, and may be used in any medical, dental, surgical/orthopaedic, or veterinarian application through appropriate extension.

### REFERENCE TO RELATED APPLICATION

This is a divisional of U.S. application Ser. No. 10/410,026, filed Apr. 9, 2003, which claims the benefit of U.S. application Ser. No. 60/371,377, filed Apr. 10, 2002, both of which are expressly incorporated herein by reference in their entirety.

### FIELD OF THE INVENTION

This invention relates generally to spinal surgery and, in particular, to instrumentation and methods that aid in determining the risk of extrusion of intradiscal devices.

### BACKGROUND OF THE INVENTION

Several hundred thousand patients undergo disc operations each year. Approximately five percent of these patients will suffer recurrent disc herniation, which results from a void or defect which remains in the outer layer (annulus fibrosis) of the disc after surgery involving partial discectomy.

In the case of a healthy anatomy, the nucleus pulposus is entirely surrounded by the annulus fibrosis. FIG. 1 illustrates the case of the herniated disc, wherein a portion of the nucleus pulposus has ruptured through a defect in the annulus fibrosis. FIG. 2 illustrates a post-operative anatomy following partial discectomy, wherein a space remains adjacent a hole or defect in the annulus fibrosis following removal of the disc material. The hole may act as a pathway for additional material to protrude into surrounding tissues, including the nerve, resulting in the recurrence of the herniation. Thousands of patients each year require surgery to treat this condition, often involving the introduction or implantation of a device into the intervertebral disc space.

However, therapeutic devices placed in human intervertebral discs often extrude through the hole in the disc through which the device was placed. Extrusion of such materials is estimated to be as high as 50 percent in some cases. Extrusion of artificial disc replacements can cause injury to a patient's nerves, spinal cord, or other surrounding structures. Pressure on a patient's nerves can be extremely painful. Patients with extruded artificial disc replacements often undergo secondary operations to remove or reposition the disc replacement.

### SUMMARY OF THE INVENTION

This invention is directed to instrumentation for determining the integrity of a natural or synthetic implant. In broad and general terms the apparatus includes a component that is at least temporarily attached to or constrained by a bone or tissue, and a handle coupled to the component including a force-measurement gauge. Operation of the device permits a user to push or pull on the handle and determine the integrity of the component by referring to the force-measurement gauge. The handle may be modularly attachable to a plurality of different components or the same component at different sizes for different uses.

One preferred embodiment resides in a disc pressure gauge that helps in determining the risk of extrusion of intradiscal devices. In such a case, the component may be a piece of deformable material, and the gauge indicates the pressure associated with pulling (or pushing) the material through a defect in the annulus fibrosis, for example. In alternative embodiments, the handle may be coupled to other natural or artificial components, including a nucleus replacement or other intradiscal device, fusion plug, vertebral endplate or other type of plate, a pedicle screw or other type of screw. Although described in terms of spinal applications, the invention is not limited in this regard, and may be used in any medical, dental, surgical/orthopaedic, or veterinarian application through appropriate extension.

### BRIEF DESCRIPTION OF THE DRAWINGS

FIG. 1 is a simplified cross-section of an intervertebral disc shown the way in which a herniated nucleus pulposus may protrude through a defect in the annulus fibrosis;

FIG. 2 is a drawing of the disc of FIG. 1 following the removal of the herniation;

FIG. 3 is a drawing of a pressure gauge according to this invention, depicted in partial cross-section;

FIG. 4 is a drawing which illustrates the insertion of the gauge of FIG. 3 into an intervertebral disc;

FIG. 5 shows the way in which the shaped end of the gauge returns to a non-compressed state following insertion;

FIG. 6 shows how the gauge will remain in place despite a relatively large external pulling force;

FIG. 7 shows how the gauge will be expelled through the defect in the annulus in some cases with relatively little applied external force;

FIG. 8 is an axial cross section of a disc, a nucleus replacement (NR), and an alternative embodiment of a gauge;

FIG. 9 is a sagittal cross section of the spine, a cage, and the embodiment of the gauge drawn in FIG. 8;

FIG. 10 is a sagittal cross section of the spine, a total disc replacement (TDR), and the embodiment of the gauge drawn in FIG. 8;

FIG. 11 is a sagittal cross section of the spine, a TDR, and the embodiment of the gauge drawn in FIG. 8;

FIG. 12 is a sagittal cross section of a vertebra, a pedicle screw, and a gauge according to the invention;

FIG. 13 is a sagittal cross section of the spine, a plate and screws, and a gauge according to the invention;

FIG. 14 a sagittal cross section of the spine, a fusion graft, and a gauge according to the invention;

FIG. 15A is a sagittal cross section of an alternative modular gauge;

FIG. 15B is a sagittal cross section of a disc and the intradiscal component of the embodiment of the gauge drawn in FIG. 15A; and

FIG. 15C is a sagittal cross section of a disc, and the embodiment of the gauge drawn in FIG. 15A.

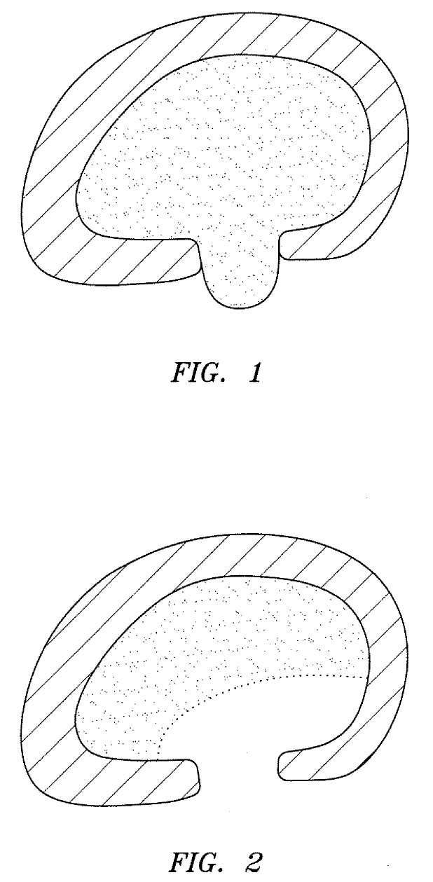

### DETAILED DESCRIPTION OF THE INVENTION

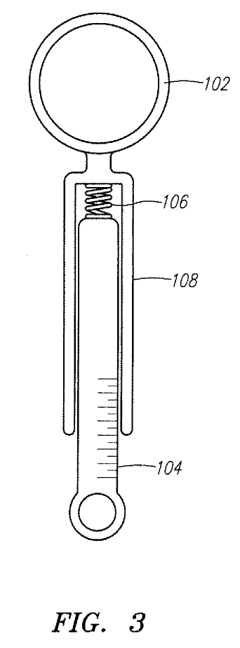

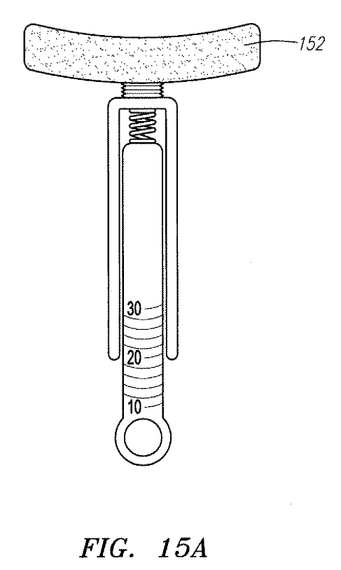

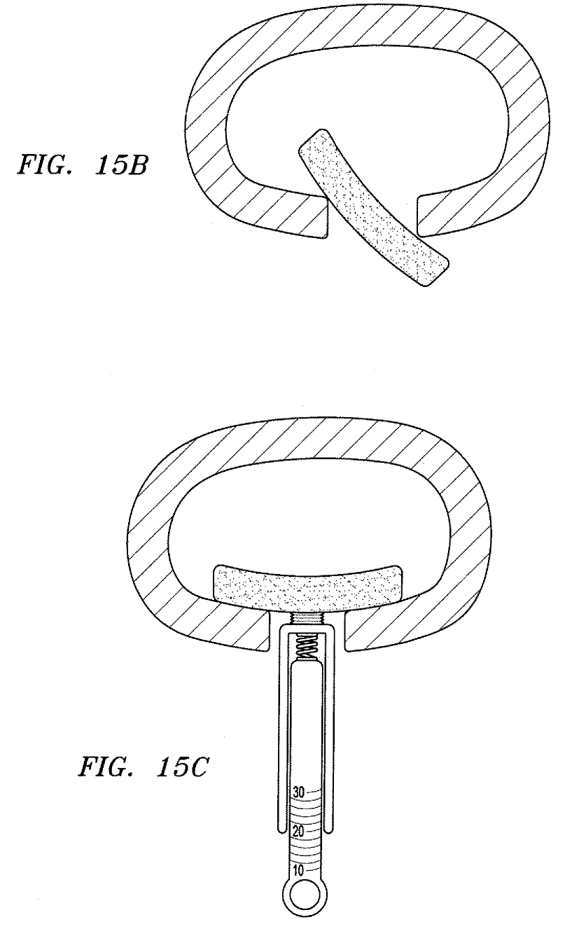

This invention is directed to implant integrity measurement apparatus finding utility in numerous applications. One preferred embodiment resides in a disc pressure gauge that helps in determining the risk of extrusion of intradiscal devices. Broadly, this embodiment comprises a piece of shaped material having one or more predetermined spring-like properties. In the preferred embodiment, a ring of metal 102 is used, as shown in FIG. 3. Alternatively, the intradiscal component of the tool could also be a balloon that inflates and deflates, similar to balloon catheters, or a shaped form like that shown in FIG. 15.

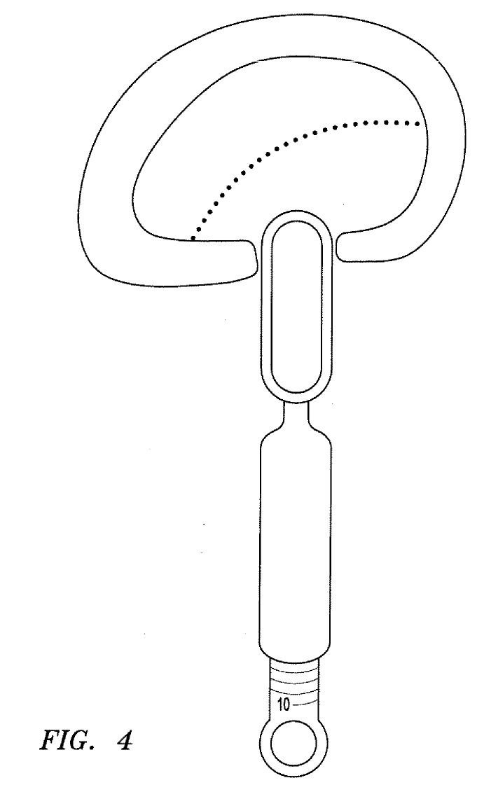

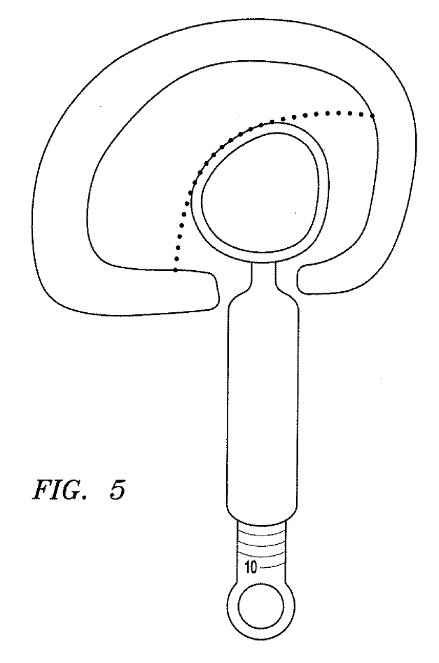

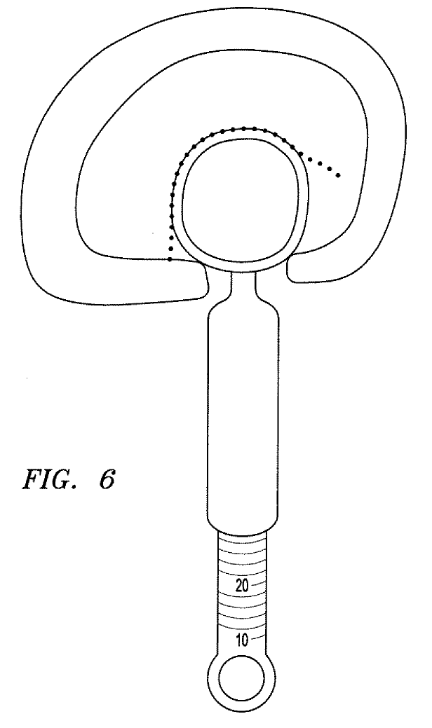

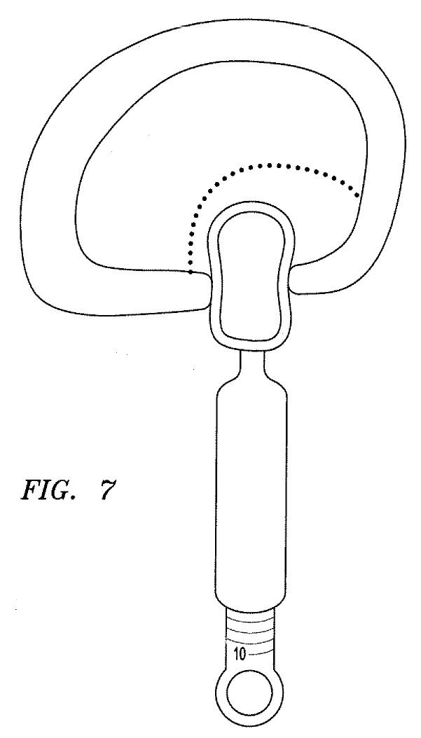

In an elongated form, the device can be forced through a hole in the annulus fibrosis of the disc, as shown in FIG. 4. Once inside the disc, the ring returns to a circular (or other predetermined) shape, as shown in FIG. 5. Pressure is then applied to try to pull the ring out through the hole in the annulus fibrosis. The ring changes shape as external pressure is applied.

The amount of force required to pull the ring through the opening in the annulus is measured by a scale 104 attached to a spring 106 connected to the ring within the body of a handle 108. If the surgeon can pull the ring through the annular ring with a small amount of force, intradiscal devices are at a high risk of extrusion. Conversely, if the ring remains in the disc space despite a large amount of force, intradiscal devices are less likely to extrude. For example, the gauge pulls out with a relatively low pressure, such as 16 in/lbs, this may signify a more serious problem than if the gauge remains in place despite a higher pressure such as 30 in/lbs. It will be appreciated that the ring 102 or other piece of shaped material may be removable, allowing different sized units to be fitted thereon, perhaps with different degrees of deformational integrity to suit various patient anatomies.

FIG. 8 is an axial cross section of a disc, a nucleus replacement (NR), and an alternative embodiment of the gauge. The gauge may be reversible attached to an intradiscal device. For example, the gauge may be screwed into a NR. The gauge would be removed after measuring the “pull-out strength” of the NR.

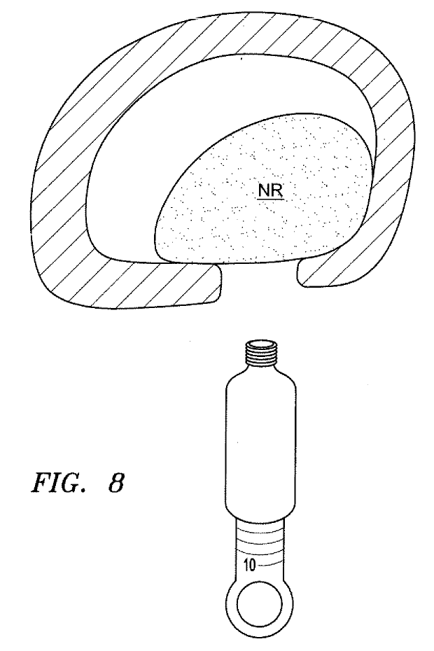

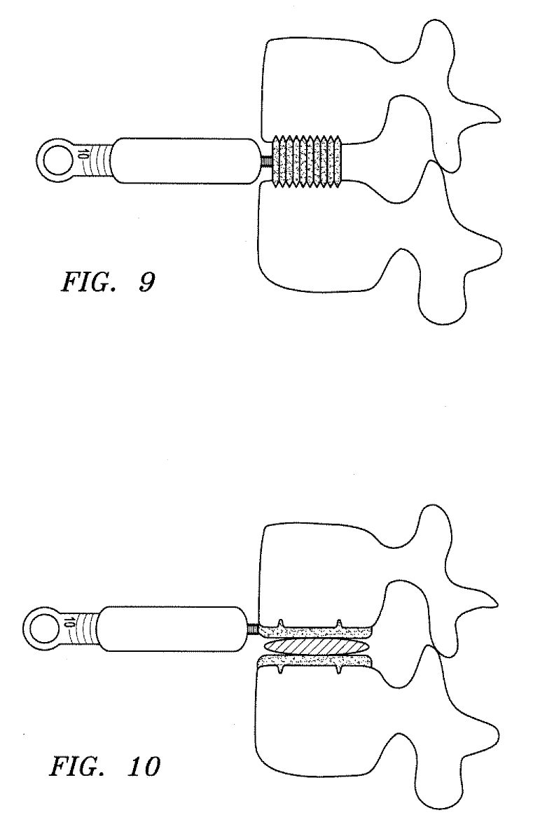

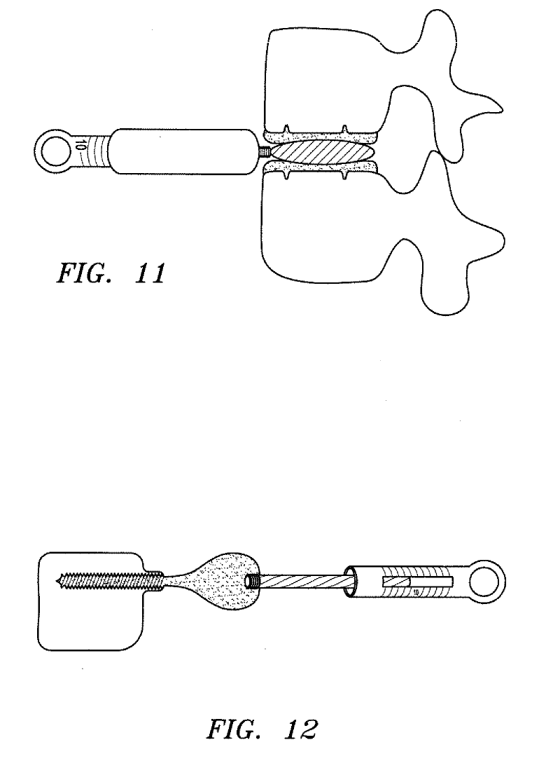

FIG. 9, is a sagittal cross section of the spine, a cage, and an embodiment of a gauge that is reversibly attached to the cage. The gauge measures the “pull-out strength” of the cage. FIG. 10 is a sagittal cross section of the spine, a total disc replacement (TDR), and a gauge reversibly attached to an endplate of the TDR. FIG. 11 is a sagittal cross section of the spine, a TDR, and the embodiment of the gauge drawn in FIG. 8. The gauge is reversibly attached to a TDR component between the TDR endplates.

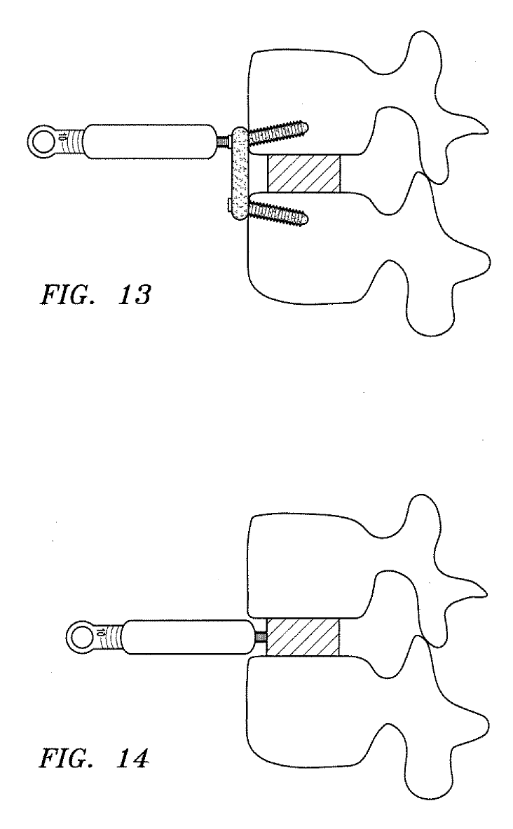

FIG. 12 is a sagittal cross section of a vertebra, a pedicle screw, and a gauge reversibly attached to the pedicle screw. The gauge measures the resistance of the screw to “pull-out.” FIG. 13 is a sagittal cross section of the spine, a plate and screws, and gauge reversibly attached a vertebral screw. The gauge measures the “pull-out strength” of the screw. The gauge can also be reversibly attached to the plate to measure “pull out strength” of the plate/screw(s) combination.

FIG. 14 is a sagittal cross section of the spine, a fusion graft, and the novel gauge. The gauge is reversibly attached to the graft. The graft could be made of bone or bone inducing material. The gauge is reversibly attached to the graft.

FIG. 15A is a sagittal cross section of an alternative modular embodiment a gauge. The intradiscal component 152 of the gauge is reversibly attached to the gauge. In FIG. 15B, the intradiscal component of the gauge is drawn during its insertion into a hole in the Annulus Fibrosis (AF). In FIG. 15C, the measuring component of the gauge has been reversibly connected to the intradiscal component of the gauge. The gauge is disassembled and removed after measuring the “pull-out strength” provided by the AF.

What is claimed is:

1. An instrument for determining the integrity of a natural or synthetic implant, comprising:
a deformable ring;
a handle coupled to the deformable ring, the handle and deformable ring arranged along a common axis; and
a force-measuring gauge operably connected to the deformable ring and the handle to measure a force along the axis, the force being one that stretches the handle from the deformable ring or compresses the handle and the deformable ring to enable a user to determine the force required to pass the deformable ring through a defect in a bone or tissue.

2. The instrument of claim 1, wherein the gauge indicates the pressure associated with passing the component through a defect in an annulus fibrosis.

3. The instrument of claim 1, wherein the handle is modularly attachable to a plurality of different deformable components for different uses.
